(19) United States
(12) Patent Application Publication
(10) Pub. No.: US 2022/0107317 A1
(43) Pub. Date: Apr. 7, 2022

(54) COMPOSITIONS AND METHODS FOR DIAGNOSING AND TREATING CORONAVIRUS DISEASE 2019

(71) Applicant: PD BIOMEDICAL CO., LTD., Taoyuan City (TW)

(72) Inventors: Chia-Ching Chang, Hsinchu (TW); Chia-Yu Chang, Hsinchu (TW); Shin-Ru Shih, Hsinchu (TW); Sheng-Yu Huang, Hsinchu (TW)

(21) Appl. No.: 17/491,568

(22) Filed: Oct. 1, 2021

Related U.S. Application Data
(60) Provisional application No. 63/088,503, filed on Oct. 7, 2020.

(51) Int. Cl. G01N 33/569; G01N 33/543; G01N 33/544

(52) U.S. Cl. CPC G01N 33/56983; G01N 2800/26; G01N 33/544; G01N 33/54306

(57) ABSTRACT

Provided are methods and compositions for the diagnosis and treatment of COVID-19, a disease caused by SARS-CoV-2 infection. More specifically, peptides that bind to SARS-CoV-2 are provided for use as diagnostic and therapeutic compositions in diagnosis, treatment and prevention of individuals contracting, or in danger of contracting COVID-19.

### BACKGROUND

### 1. Technical Field

The present disclosure relates to the fields of molecular biology, protein chemistry, immunochemistry and pharmacology in describing methods and compositions for the diagnosis and treatment of coronavirus disease 2019 (COVID-19), a disease caused by infection of severe acute respiratory syndrome coronavirus 2 (SARS-CoV-2).

### 2. Description of Related Art

Coronavirus disease 2019 (COVID-19) is an infectious disease caused by severe acute respiratory syndrome coronavirus 2 (SARS-CoV-2). Since first identified in December 2019 in Wuhan, Hubei, China, the disease has caused a worldwide pandemic. The World Health Organization (WHO) declared the COVID-19 outbreak a public health emergency of international concern (PHEIC) on January 30, 2020 and a pandemic on March 11, 2020. As of September 2020, more than 29.1 million cases have been reported across 188 countries and territories with more than 927,000 deaths.

For an efficient containment of a pandemic disease, accurate and fast diagnosis is important to identify patients at the earliest time for isolation. The standard method of diagnosis is by real-time reverse transcription polymerase chain reaction (rRT-PCR) from a nasopharyngeal swab. However, carrying out the test by rRT-PCR involves procedures such as nucleic acid extraction and gene amplification, which require trained personnel, specific chemical supplies and expensive instruments that are often available only in labs that provide routine, centralized services, and take hours to provide results. This limits the number of tests that can be done, especially in developing countries. Furthermore, due to the complicated procedures, accuracy of rRT-PCR can be affected by loss of samples during nucleic acid extraction, or the efficiency of polymerase synthesis. Therefore, a faster and easy-to-manage method for diagnosing COVID-19 is in pressing need.

Furthermore, there are no effective treatments for COVID-19 yet. An effective treatment or preventive measure for COVID-19 remains a common goal of the scientists and researchers worldwide.

### SUMMARY

The present disclosure relates to a use of teneurin-2 (TENM2) polypeptides and fragments thereof as a diagnostic molecule for detecting COVID-19 and identifying subjects infected with SARS-CoV-2. The present disclosure also relates to a use of teneurin-2 (TENM2) polypeptides and fragments thereof as compositions to treat COVID-19. Another aspect of the present disclosure relates to prevention of SARS-CoV-2 infection using teneurin-2 (TENM2) polypeptides and fragments thereof.

Accordingly, methods for detecting and diagnosing COVID-19, identifying subjects infected with SARS-CoV-2, treating COVID-19 and preventing infection caused by SARS-CoV-2 and thereby preventing COVID-19 are provided.

In accordance with the methods of the present disclosure, a non-natural polypeptide comprising a fragment of teneurin-2 (TENM2) is provided.

In at least one embodiment, a method of the present disclosure for detecting SARS-CoV-2 in a subject in need thereof comprises providing a biological sample from the subject; contacting a non-natural polypeptide including a fragment of teneurin-2 (TENM2) to the biological sample; and detecting binding activity of the non-natural polypeptide. In at least one embodiment, the non-natural polypeptide includes the extracellular domain of TENM2, or a fragment thereof. In at least one embodiment, the extracellular domain of TENM2 has an amino acid sequence of SEQ ID NO: 6. In at least one embodiment, the non-natural polypeptide has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to the extracellular domain of TENM2.

In at least one embodiment, the fragment of the extracellular domain of TENM2 comprises at least one amino acid sequence selected from SEQ ID NO: 2, SEQ ID NO: 3, SEQ ID NO: 4, SEQ ID NO: 13, SEQ ID NO: 14 and SEQ ID NO: 15 or any combination thereof. In at least one embodiment, the fragment of the extracellular domain of TENM2 comprises a peptide having at least 50%, at least 55%, at least 60%, at least 65%, at least 70%, at least 75%, at least 80%, at least 85%, at least 90%, at least 92%, at least 93%, at least 95%, at least 96%, at least 97%, at least 98% or at least 99% sequence identity to SEQ ID NO: 2, SEQ ID NO: 3, SEQ ID NO: 4, SEQ ID NO: 13, SEQ ID NO: 14 and SEQ ID NO: 15 and shows binding activity to SARS-CoV-2 spike protein receptor binding domain. In at least one embodiment, the fragment of the extracellular domain of TENM2 has an amino acid sequence of SEQ ID NO: 5, SEQ ID NO: 7, SEQ ID NO: 8, SEQ ID NO: 9, SEQ ID NO: 10, SEQ ID NO: 11, SEQ ID NO: 12 or SEQ ID NO: 16. In at least one embodiment, the non-natural polypeptide has at least 75%, at least 80%, at least 85%, at least 90%, at least 91%, at least 92%, at least 93%, at least 94%, at least 95%, at least 96%, at least 97%, at least 98% or at least 99% sequence identity to SEQ ID NO: 5, SEQ ID NO: 7, SEQ ID NO: 8, SEQ ID NO: 9, SEQ ID NO: 10, SEQ ID NO: 11, SEQ ID NO: 12 or SEQ ID NO: 16.

In at least one embodiment, the non-natural polypeptide has a length of 7 amino acids to 2,000 amino acids. In at least one embodiment, the non-natural polypeptide consists of 10 to 120 amino acids, 10 to 150 amino acids, 10 to 200 amino acids, 20 to 120 amino acids, 20 to 150 amino acids, 20 to 200 amino acids, 30 to 160 amino acids, 30 to 180 amino acids, or 30 to 200 amino acids. In at least one embodiment, the non-natural polypeptide consists of 60 to 150 amino acids or 60 to 120 amino acids. In at least one embodiment, the non-natural polypeptide consists of 90 to 120 amino acids.

In at least one embodiment, the method for detecting SARS-CoV-2 in a subject in need thereof comprises providing a biological sample from the subject; contacting a non-natural polypeptide including a fragment of teneurin-2 (TENM2) to the biological sample; and detecting binding activity of TENM2 by detecting an interaction between the non-natural polypeptide and SARS-CoV-2 by at least one of electrochemical impedance spectroscopy, immunoassay, counter immuno-electrophoresis, radioimmunoassay, radioimmunoprecipitation assay, enzyme-linked immunosorbent assay, dot blot assay, inhibition of competition assay and sandwich assay. In at least one embodiment, detecting binding activity of TENM2 to SARS-CoV-2 involves immobilization of the non-natural polypeptide including a fragment of teneurin-2 (TENM2) to palladium nano-thin-film polyethylene terephthalate (Pd NTF-PET).

In at least one embodiment, the biological sample in the method for detecting SARS-CoV-2 is a nasal discharge, secretion from respiratory tract, stool sample, or blood sample.

In another aspect of the present disclosure, a kit for detecting SARS-CoV-2 is provided. In at least one embodiment, the kit comprising a non-natural polypeptide including a fragment of teneurin-2 (TENM2) for binding SARS-CoV-2. In at least one embodiment, the kit further comprises a reagent for detecting the binding activity between the fragment of TENM2 and SARS-CoV-2. In at least one embodiment, the non-natural polypeptide provided in the kit includes the extracellular domain of TENM2, or a fragment thereof. In at least one embodiment, the non-natural polypeptide has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% sequence identity to the extracellular domain of TENM2. In at least one embodiment, the fragment of the extracellular domain of TENM2 comprises at least one amino acid sequence selected from SEQ ID NO: 2, SEQ ID NO: 3, SEQ ID NO: 4, SEQ ID NO: 13, SEQ ID NO: 14 and SEQ ID NO: 15, or any combination thereof. In at least one embodiment, the fragment of the extracellular domain of TENM2 comprises a peptide having at least 50%, at least 55%, at least 60%, at least 65%, at least 70%, at least 75%, at least 80%, at least 85%, at least 90%, at least 92%, at least 93%, at least 94%, at least 95%, at least 96%, at least 97%, at least 98% or at least 99% sequence identity to SEQ ID NO: 2, SEQ ID NO: 3, SEQ ID NO: 4, SEQ ID NO: 13, SEQ ID NO: 14 and SEQ ID NO: 15 and shows binding activity to SARS-CoV-2 spike protein receptor binding domain. In at least one embodiment, the fragment of the extracellular domain of TENM2 has an amino acid sequence of SEQ ID NO: 5, SEQ ID NO: 7, SEQ ID NO: 8, SEQ ID NO: 9, SEQ ID NO: 10, SEQ ID NO: 11, SEQ ID NO: 12 or SEQ ID NO: 16. In at least one embodiment, the non-natural polypeptide has at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to SEQ ID NO: 5, SEQ ID NO: 7, SEQ ID NO: 8, SEQ ID NO: 9, SEQ ID NO: 10, SEQ ID NO: 11, SEQ ID NO: 12 or SEQ ID NO: 16.

In another aspect of the present disclosure, a non-natural polypeptide comprising a fragment of teneurin-2 (TENM2) having at least 75% sequence identity to SEQ ID NO: 5, SEQ ID NO: 7, SEQ ID NO: 8, SEQ ID NO: 9, SEQ ID NO: 10, SEQ ID NO: 11, SEQ ID NO: 12 or SEQ ID NO: 16 is provided. In at least one embodiment, the non-natural polypeptide has at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity to SEQ ID NO: 5, SEQ ID NO: 7, SEQ ID NO: 8, SEQ ID NO: 9, SEQ ID NO: 10, SEQ ID NO: 11, SEQ ID NO: 12 or SEQ ID NO: 16. Also provided is a pharmaceutical composition comprising the non-natural polypeptide and a pharmaceutically acceptable carrier thereof.

Another aspect of the present disclosure provides a use of the above non-natural polypeptides in the prevention of SARS-CoV-2 infection.

### BRIEF DESCRIPTION OF THE DRAWINGS

The present disclosure will become more readily appreciated by reference to the following descriptions in conjunction with the accompanying drawings.

FIG. 1 shows the platform and steps used to screen the peptides binding to the receptor binding domain (RBD) of the spike protein of SARS-CoV-2.

FIG. 2 illustrates the binding test between SARS-CoV-2 and candidate SARS-CoV-2 binding peptides by electrochemical impedance spectroscopy (EIS)-based bio-sensing platform with the candidate SARS-CoV-2 binding peptides immobilized on a palladium nano-thin-film on polyethylene terephthalate (Pd NTF-PET) substrate.

FIG. 3 shows the result of binding between SARS-CoV-2 and T2 peptide at different concentrations of SARS-CoV-2 spike protein. A Rct ratio on the y-axis is the impedance reading collected by the EIS-bio-sensing platform, indicating the binding between SARS-CoV-2 spike protein and T2 peptide.

FIG. 4 shows the western blot showing T2 peptide used to detect SARS-CoV-2 pseudovirus.

FIG. 5 shows the amount of luciferase activity as an indicator of SARS-CoV-2 pseudovirus level in OECM1 cells after adding SARS-CoV-2 pseudovirus incubated without or with different amounts of binding peptide T2.

### DETAILED DESCRIPTIONS

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the above examples for carrying out this disclosure without contravening its scope for different aspects and applications.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein have to be defined based on the meaning of the terms together with the descriptions throughout the specification.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

Also, when a part “includes” or “comprises” a component or a step, unless there is a particular description contrary thereto, the part can further include other components or other steps, not excluding the others.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.” The term “about” means plus or minus 0.1% to 50%, 5% to 50%, 10% to 40%, 10% to 20%, or 10% to 15% of the number to which reference is being made.

The term “peptide” used herein refers to a short chain containing more than one amino acid monomers, in which the more than one amino acid monomers are linked to each other by amide bonds. It is to be noted that the amino acid monomers used in the peptide of the present disclosure are not limited to natural amino acids, and the amino acid sequence of the peptide can also include unnatural amino acids, compounds with similar structures, or the deficiency of amino acids.

The terms “polypeptide” and “peptide” are used interchangeably herein to refer to polymers of amino acids of any length. The polymer may be linear or branched. It may comprise modified amino acids, and may be interrupted by non-amino acids. The terms also encompass an amino acid polymer that has been modified, e.g., disulfide bond formation, glycosylation, lipidation, acetylation, phosphorylation, or any other manipulation, such as conjugation with a labeling component. The polypeptide can be isolated from natural sources, can be produced by recombinant techniques from a eukaryotic or prokaryotic host, or can be a product of synthetic procedures.

It is understandable that a polypeptide may have a limited number of changes or modifications that may be made within a certain portion of the polypeptide irrelevant to its activity or function and still result in a variant with an acceptable level of equivalent or similar biological activity or function. The term “acceptable level” can mean at least 20%, at least 50%, at least 60%, at least 70%, at least 80%, or at least 90% of the level of the referenced protein as tested in a standard assay as known in the art. Biologically functional variant polypeptides are thus defined herein as those polypeptides in which certain amino acid residues may be substituted. Polypeptides with different substitutions may be made and used in accordance with this disclosure. Modifications and changes may be made in the structure of such polypeptides and still obtain a molecule having similar functions. For example, certain amino acids may be substituted for other amino acids in the peptide/polypeptide structure without appreciable loss of activity. Variants can be prepared according to methods for altering a polypeptide sequence known to one of ordinary skill in the art, such as those are found in references which compile such methods, e.g., “Molecular Cloning: A Laboratory Manual,” J. Sambrook, et al., eds., Second Edition, Cold Spring Harbor Laboratory Press, Cold Spring Harbor, New York, 1989. For example, conservative substitutions of amino acids include substitutions made amongst amino acids within the following groups: (i) Ala, Gly; (ii) Ser, Thr; (iii) Gln, Asn; (iv) Glu, Asp; (v) Met, Ile, Leu, Val; (vi) Phe, Tyr, Trp; and (vii) Lys, Arg, His.

Peptides used herein may be isolated from a variety of sources, such as from human tissue types or from other sources, or prepared by recombinant or synthetic methods, or by any combination of these and similar techniques. Peptide variants include peptides comprising amino acid sequences sufficiently identical to or derived from the amino acid sequence of a native peptide which includes fewer amino acids than the native peptides. A portion or a fragment of a peptide can be a peptide which is, for example, 3 to 5, 8 to 10, 10, 15, 15 to 20, 20, 25, 30, 35, 40, 45, 50, 60, 70, 80, 90, 100, 110, 120, 130, 150, 200, 300 or more amino acids in length. Portions or fragments in which regions of a polypeptide are deleted can be prepared by recombinant techniques and can be evaluated for one or more functional activities such as the ability to form antibodies specific to a peptide. A portion or a fragment of a peptide may comprise a domain of the native peptide or a portion or a fragment of such domain.

As used herein, the term “sequence identity” or, for example, comprising a “sequence having 80% sequence identity with,” as used herein, refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 80%, 85%, 90%, 95%, 97%, 98%, 99% or 100% sequence identity to any of the reference sequences described herein (see, e.g., Sequence Listing), typically where the polypeptide variant maintains at least one biological activity or function of the reference polypeptide.

The term “detect,” “detecting” or “detection” includes assaying, or otherwise establishing the presence or absence of the target molecule(s), protein domain(s), subunits, or combinations of reagent-bound targets, and the like.

The terms “subject,” “patient” and “individual” are used interchangeably herein and refer to a warm-blooded animal such as a mammal that is afflicted with, or suspected of having, at risk for or being pre-disposed to, or being screened for viral infection, including actual or suspected SARS-CoV-2 infection. These terms include, but are not limited to, domestic animals, sports animals, primates and humans. For example, the terms refer to a human.

The term “biological sample” refers to a sample to be analyzed by any of the methods described herein that can be of any type of samples obtained from a subject to be detected. The biological samples used herein include, but are not limited to: nasal discharge, secretion from respiratory tract, blood, serum, plasma, urine, sputum, saliva, cerebrospinal fluid, interstitial fluid, mucous, sweat, stool extract, fecal matter, synovial fluid, tears, semen, peritoneal fluid, nipple aspirates, milk, vaginal fluid, or any combination thereof. In some embodiments, a blood sample can be whole blood or a faction thereof, e.g., serum or plasma, heparinized or EDTA treated to avoid blood clotting.

As used herein, the terms “therapies” and “therapy” can refer to any protocol(s), method(s), composition(s), formulation(s), and/or agent(s) that can be used in the prevention or treatment of a disease or symptom associated therewith. In at least one embodiment, the terms “therapies” and “therapy” refer to biological therapy, supportive therapy, and/or other therapies useful in prevention or treatment of a disease or symptom associated therewith known to one of ordinary skill in the art.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen) to delay or prevent disease occurrence. Treatment may also be continued after symptoms have resolved, for example, to delay or prevent recurrence.

As used herein, the term “preventing” or “prevention” refers to preventive or avoidance measures for a disease or symptoms or conditions of a disease, which include but are not limited to applying or administering one or more active agents to a subject who has not yet been diagnosed as a patient suffering from the disease or the symptoms or conditions of the disease but may be susceptible or prone to the disease. The preventive measures are used to avoid, prevent, or postpone the occurrence of the disease or the symptoms or conditions of the disease.

The kits provided herein are in suitable packaging. Suitable packaging includes, but is not limited to, vials, bottles, jars, flexible packaging, and the like. Also contemplated are packages for use in combination with a medical device, such as an inhaler, nasal administration device, or an infusion device. A kit may have a sterile access port (for example, the container may be an intravenous solution bag or a vial having a stopper pierceable by a hypodermic injection needle). The container may also have a sterile access port. Kits optionally may provide additional components such as buffers and interpretive information. Normally, the kit comprises a container and a label or package insert(s) on or associated with the container. In some embodiments, the disclosure provides articles of manufacture comprising contents of the kits described above.

As used herein, the term “pharmaceutical composition” or “pharmaceutical combination” can be prepared according to any method known to the art for the manufacture of pharmaceuticals. Such composition or combination may contain sweetening agents, flavoring agents, coloring agents and preserving agents. A formulation can be admixed with nontoxic pharmaceutically acceptable excipients which are suitable for manufacture. Non-limiting formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, lozenges, packets, troches, elixirs, suspensions, solutions, syrups, soft and hard gelatin capsules, suppositories, sterilized injection fluid, packaged powder, on patches, in implants, etc.

As used herein, pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; physiological saline; sterilized water; isotonic agents; and/or non-ionic surfactants. See, e.g., Remington: The Science and Practice of Pharmacy 20th Ed. (2000) Lippincott Williams and Wilkins, Ed. K. E. Hoover.

### EXAMPLES

Exemplary embodiments of the present disclosure are further described in the following examples, which should not be construed to limit the scope of the present disclosure.

### Example 1. Screening of Peptides Binding to the Receptor Binding Domain of SARS-CoV-2 Spike Protein

To screen for the peptides that bind to the receptor binding domain (RBD) of SARS-CoV-2 spike protein (SARS-CoV-2 spike protein RBD), a technique named phage display is adopted. Phage display is a technology for rapid discovery of novel peptides binding with target proteins from an artificial random peptide library. The phage library used here includes peptides with 12 amino acids that are randomly arranged, which was obtained from New England Biolab. Co.

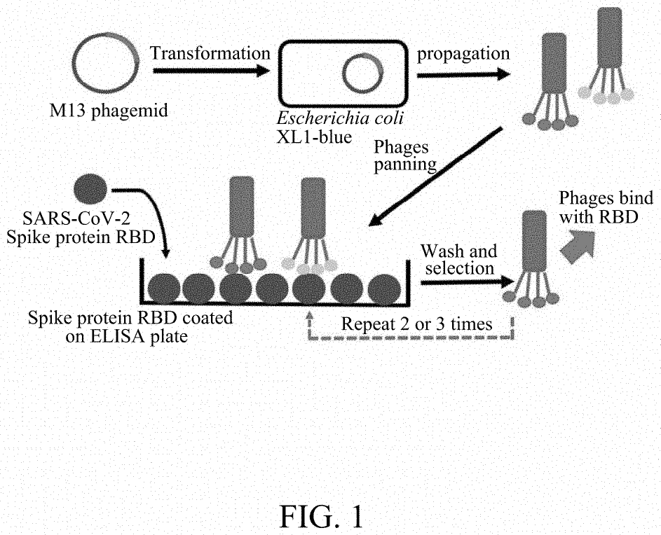

As shown in FIG. 1, SARS-CoV-2 spike protein RBD was first expressed by E. coli and purified to be immobilized on the enzyme-linked immunosorbent assay (ELISA) plate for 2 to 3 hours at room temperature. Then, the phage library mixture containing collection of artificial random peptides was added to the ELISA plate coated with SARS-CoV-2 spike protein RBD. The phage library mixture titer reaches 10⁷ to 10⁸ phages/mL. After 12 hours of phages and spike protein RBD panning at 4° C., the excess phage mixture was discarded from plate holes, and each plate hole was washed with phosphate-buffered saline (PBS) plus 0.1% Tween 20. The washing procedure was repeated 3 times. Then, the phage bound to spike protein RBD was eluted with 0.5 M glycine buffer from each plate holes. Subsequently, the phage eluted was mixed with E. coli XL1 blue (0D₆₀₀ around 0.5) and incubated at 37° C. for 8 hours. Then, the E. coli cells were spun down, and the bacterial medium was concentrated with 1.5 volume of 2% polyethylene glycol (PEG) 600 plus 250 mM sodium chloride buffer. The concentrated medium thus obtained contained 1-round of panning phages. Using the obtained 1-round panning phages to bind with the new ELISA plate with immobilized spike protein RBD again. The procedure was repeated for a total of 3 times to obtain the 3-round panning phages.

Finally, the mixture of 3-round panning phages was used to infect the E. coli XL1-blue and incubated for 3 hours. Then, the bacteria were plated on Luria-Bertani (LB) agar containing 1 mM of isopropyl β-D-thiogalactoside (IPTG) and 20 μg/mL of 5-bromo-4-chloro-3-indolyl-β-D-galactopyranoside (X-GAL). After overnight incubation, the blue colored colonies were picked up, and the phagemids were purified, followed by check of pattern consistency of the phagemids. Then, the phagemids were sequenced to confirm the spike protein RBD binding peptide.

### Example 2. Identification of a SARS-CoV-2 Spike Protein RBD Binding Peptide

Following the phage display method described above, a phagemid displaying a peptide having an amino acid sequence of MKKLLFAIPLVVPFYSHSTAAATITQSTIPGGGSAETVESCLAKSHTENSFTNV WKDDKTLDRYANYEGCLWNATGVVVCTGDETQCYGTWVPIGLAIPENEGG GSEGGGSEGGGSEGGGTKPPEYGDTPIPGYTYINPLDGTYPPGTENSG (SEQ ID NO: 1) was identified as a peptide binding to SARS-CoV-2 spike protein RBD. In the peptide, the peptide fragment having an amino acid sequence of TAAATITQSTIP (SEQ ID NO: 2) was identified as the peptide fragment binding to SARS-CoV-2 spike protein RBD. From this peptide fragment binding to SARS-CoV-2 spike protein RBD, the peptide having an amino acid sequence of TITQSTIP (SEQ ID NO: 3) was blasted against known proteins on the website of National Center for Biotechnology Information (NCBI), and it was found to have a 75% sequence identity to a sequence fragment of the extracellular domain of human teneurin-2 protein (TENM2), i.e., TMTQSTVP (SEQ ID NO: 17).

An artificial sequence fragment named as T2 peptide was designed based on the identified fragment of TENM2, which has the amino acid sequence of DLSGFVRPDPVIISSPLSTFFSDAGYKSLLKITMTQSTVPLNLIRVHLMVAVEG HLFQKSFQASPNLAYTFIWDKTDAYGQRVYGLSDAVVSVGFEYETCPSLILW EKRTALLQGFELD (SEQ ID NO: 5).

Based on a comparison of conserved regions of the TENM2 sequence, other fragments of TENM2 having similar amino acid sequence and functions were obtained, including:

(SEQ ID NO: 8)
MDLSGFVRPDPVIISSPLSTFFSDAGYKSLLKITMTQSTVPLNLIRVHLM
VAVEGHLFQKSFQASPNLAYTFIWDKTDAYGQRVYGLSDAVVSVGFEYET
CPSLILWEKRTALLQGFELD;
(SEQ ID NO: 9)
TQVLHEEIELPGSNVKLRYLSSRTAGYKSLLKITMTQSTVPLNLIRVHLM
VAVEGHLFQKSFQASPNLAYTFIWDKTDAYGQRVYGLSDAVVSVGFEYET
CPSLILWEKRTALLQGFELD;
(SEQ ID NO: 10)
TQVLHEEIEVPGSSIKLIYLSSRTAGYKSLLKIIMTQSLVPLNLIKVHLM
VAVEGHLFQKSFLASPNLAYTFIWDKTDAYGQKVYGLSDAVVSVGFEYET
CPSLILWEKRTALLQGFELD;
(SEQ ID NO: 11)
TQIVQESIQIPGSDLHLTYQSSQASGYLSIVRMRLTAETIPPTLTHVHVG
VEIEGALHVKTYEADPSLVHTFAWNKRNVYRQKVYGVTVARISVGY;
(SEQ ID NO: 12)
SQVIQESLQIPGTGLNLVYHSSRAAGYLSTIKLQLTPDVIPTSLHLIHLR
ITIEGILFERIFEADPGIKFTYAWNRLNIYRQRVYGVTTAVVKVGYQY;
and
(SEQ ID NO: 16)
TQVLHEEIELPGSNVKLRYLSSRTAGYKSLLKITMTQSTVPLNLIRVHLM
VAVEGHLFQKSFQASPNLAYTFIWDKTDAYGQRVYGLSDAVVSVGF.

### Example 3. Binding Test of T2 Peptide Towards SARS-CoV-2 Spike Protein

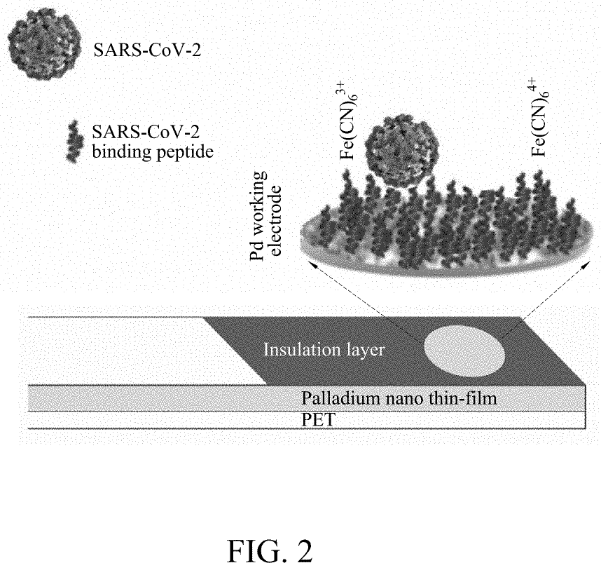

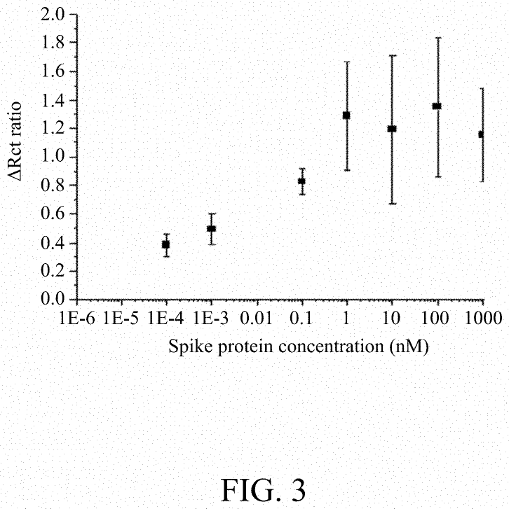

The T2 peptides were expressed by E. coli using the bacterial protein expression method well-known by a person skilled in the art. Then, the peptides were purified and immobilized on palladium nano-thin-film polyethylene terephthalate (Pd NTF-PET), which is developed for electrochemical impedance spectroscopy (EIS), as shown in FIG. 2. Peptides were immobilized on the Pd NTF-PET electrodes within 15 minutes, and its biosensing sensitivity was as low as 0.1 ng in 1 μL per test by measuring the change in impedance upon binding to SARS-CoV-2 spike protein. As shown in FIG. 3, T2 peptide showed binding activity to SARS-CoV-2 spike protein at a concentration as low as 0.0001 nM by EIS.

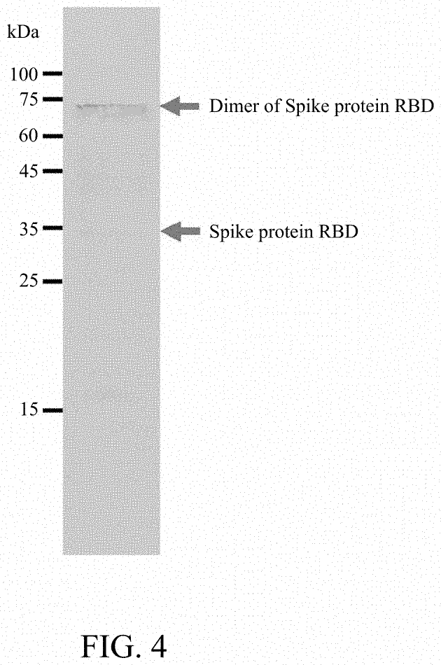

Western blot results using T2 peptide against SARS-CoV-2 spike protein RBD is shown in FIG. 4, indicating the binding between T2 peptide to both spike protein RBD and the dimer of spike protein RBD.

### Example 4. Inhibition of SARS-CoV-2 Infection by T2 Peptide

To test the inhibiting ability of T2 peptide on SARS-CoV-2 infection, nCoV-S Luc pseudovirus of SARS-CoV-2 with luciferase activity was used to incubate with T2 peptide at different relative light units (RLU) for an hour and then added to OECM1 cells. The nCoV-S Luc pseudovirus was obtained from the National RNAi Core Facility at Academia Sinica, Taiwan, which uses pCMVdeltaR8.91 and pcDNA3.1 to express S-proteins on the surface of the pseudovirus. The entry of pseudovirus can be identified by the luminescence emitted from the luciferase encoded in the transfer vector pLAS2w.FLuc.Ppuro.

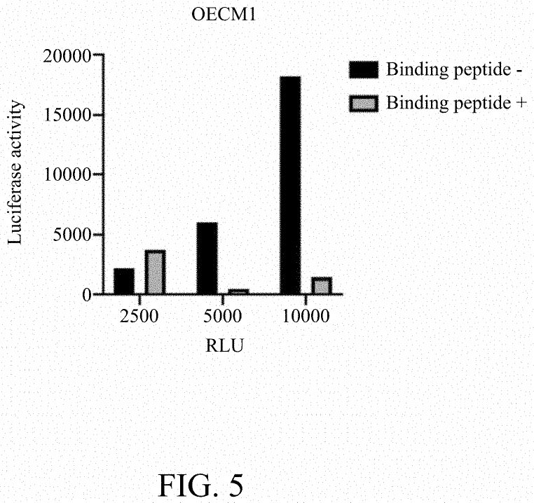

The OECM1 cells were analyzed for luciferase activity, which represented the infection resulted by the SARS-CoV-2 pseudovirus. As shown in FIG. 5, adding T2 peptide to 5,000 and 10,000 RLU of pseudovirus significantly inhibited the infection of OECM1 cells compared to the pseudovirus without prior incubation with T2 peptide.

While some of the embodiments of the present disclosure have been described above, it is, however, possible for those of ordinary skill in the art to make various modifications and changes to these embodiments shown without substantially departing from the teaching of the present disclosure. Such modifications and changes are encompassed in the scope of the present disclosure as set forth in the appended claims.

What is claimed is:

1. A method for detecting SARS-CoV-2, comprising:
providing a biological sample from a subject in need thereof;
contacting a non-natural polypeptide including a fragment of teneurin-2 (TENM2) to the biological sample; and
detecting binding activity of the non-natural polypeptide.

2. The method according to claim 1, wherein the non-natural polypeptide includes an extracellular domain of TENM2, or a fragment thereof.

3. The method according to claim 2, wherein the non-natural polypeptide has at least 75% sequence identity to the extracellular domain of TENM2.

4. The method according to claim 2, wherein the extracellular domain of TENM2 has an amino acid sequence of SEQ ID NO: 6.

5. The method according to claim 2, wherein the fragment of the extracellular domain of TENM2 comprises at least one amino acid sequence selected from SEQ ID NO: 2, SEQ ID NO: 3, SEQ ID NO: 4, SEQ ID NO: 13, SEQ ID NO: 14 and SEQ ID NO: 15, or any combination thereof, and shows binding activity to SARS-CoV-2 spike protein receptor binding domain.

6. The method according to claim 2, wherein the fragment of the extracellular domain of TENM2 comprises a peptide having at least 75% sequence identity to SEQ ID NO: 2, SEQ ID NO: 3, SEQ ID NO: 4, SEQ ID NO: 13, SEQ ID NO: 14 and SEQ ID NO: 15, and shows binding activity to SARS-CoV-2 spike protein receptor binding domain.

7. The method according to claim 1, wherein the non-natural polypeptide has at least 75% sequence identity to SEQ ID NO: 5, SEQ ID NO: 7, SEQ ID NO: 8, SEQ ID NO: 9, SEQ ID NO: 10, SEQ ID NO: 11, SEQ ID NO: 12 or SEQ ID NO: 16.

8. The method according to claim 7, wherein the non-natural polypeptide has an amino acid sequence of SEQ ID NO: 5, SEQ ID NO: 7, SEQ ID NO: 8, SEQ ID NO: 9, SEQ ID NO: 10, SEQ ID NO: 11, SEQ ID NO: 12 or SEQ ID NO: 16.

9. The method according to claim 1, wherein the non-natural polypeptide consists of 10 to 200 amino acids.

10. The method according to claim 1, wherein detecting the binding activity comprises detecting an interaction between the non-natural polypeptide and SARS-CoV-2 by at least one of electrochemical impedance spectroscopy, immunoassay, counter immuno-electrophoresis, radioimmunoassay, radioimmunoprecipitation assay, enzyme-linked immunosorbent assay, dot blot assay, inhibition of competition assay and sandwich assay.

11. The method according to claim 1, wherein the biological sample is a nasal discharge, secretion from respiratory tract, mucous, stool, or blood.

12. The method according to claim 1, wherein the non-natural polypeptide is immobilized to palladium nano-thin-film polyethylene terephthalate (Pd NTF-PET).

13. A kit for detecting SARS-CoV-2 in a sample, the kit comprising a non-natural polypeptide including a fragment of teneurin-2 (TENM2) for binding SARS-CoV-2.

14. The kit of claim 13, wherein the non-natural polypeptide includes an extracellular domain of TENM2, or a fragment thereof.

15. A non-natural polypeptide having at least 75% sequence identity to SEQ ID NO: 5, SEQ ID NO: 7, SEQ ID NO: 8, SEQ ID NO: 9, SEQ ID NO: 10, SEQ ID NO: 11, SEQ ID NO: 12 or SEQ ID NO: 16, and showing binding activity to SARS-CoV-2 spike protein receptor binding domain.

16. A pharmaceutical composition comprising the non-natural polypeptide of claim 15 and a pharmaceutically acceptable carrier thereof.

17. A method for preventing or treating SARS-CoV-2 infection in a subject in need thereof, comprising administering to the subject the non-natural polypeptide of claim 15 and a pharmaceutically acceptable carrier thereof.


SEQUENCE LISTING (17 sequences)

SEQ ID NO: 1 - protein, 152 aa, Artificial Sequence
  An artificial peptide designed to display on phage
MKKLLFAIPLVVPFYSHSTAAATITQSTIPGGGSAETVESCLAKSHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTENSG

SEQ ID NO: 2 - protein, 12 aa, Artificial Sequence
  Fragment of artificial phage display peptide
TAAATITQSTIP

SEQ ID NO: 3 - protein, 8 aa, Artificial Sequence
  Fragment of phage display peptide
TITQSTIP

SEQ ID NO: 4 - protein, 7 aa, Homo sapiens
TMTQSTV

SEQ ID NO: 5 - protein, 119 aa, Artificial Sequence
  Designed peptide for binding to target
DLSGFVRPDPVIISSPLSTFFSDAGYKSLLKITMTQSTVPLNLIRVHLMVAVEGHLFQKSFQASPNLAYTFIWDKTDAYGQRVYGLSDAVVSVGFEYETCPSLILWEKRTALLQGFELD

SEQ ID NO: 6 - protein, 1999 aa, Homo sapiens
HHHHHHHHASDTYKLVIVLNGTTFTYTTEAVDAATAEKVFKQYANDAGVDGEWTYDAATKTFTVTEASENLYFQSGSTSCADNKDNEGDGLVDCLDPDCCLQSACQNSLLCRGSRDPLDIIQQGQTDWPAVKSFYDRIKLLAGKDSTHIIPGENPFNSSLVSLIRGQVVTTDGTPLVGVNVSFVKYPKYGYTITRQDGTFDLIANGGASLTLHFERAPFMSQERTVWLPWNSFYAMDTLVMKTEENSIPSCDLSGFVRPDPIIISSPLSTFFSAAPGQNPIVPETQVLHEEIELPGSNVKLRYLSSRTAGYKSLLKITMTQSTVPLNLIRVHLMVAVEGHLFQKSFQASPNLAYTFIWDKTDAYGQRVYGLSDAVVSVGFEYETCPSLILWEKRTALLQGFELDPSNLGGWSLDKHHILNVKSGILHKGTGENQFLTQQPAIITSIMGNGRRRSISCPSCNGLAEGNKLLAPVALAVGIDGSLYVGDFNYIRRIFPSRNVTSILELRNNPAHKYYLAVDPVSGSLYVSDTNSRRIYRVKSLSGTKDLAGNSEVVAGTGEQCLPFDEARCGDGGKAIDATLMSPRGIAVDKNGLMYFVDATMIRKVDQNGIISTLLGSNDLTAVRPLSCDSSMDVAQVRLEWPTDLAVNPMDNSLYVLENNVILRITENHQVSIIAGRPMHCQVPGIDYSLSKLAIHSALESASAIAISHTGVLYITETDEKKINRLRQVTTNGEICLLAGAASDCDCKNDVNCNCYSGDDAYATDAILNSPSSLAVAPDGTIYIADLGNIRIRAVSKNKPVLNAFNQYEAASPGEQELYVFNADGIHQYTVSLVTGEYLYNFTYSTDNDVTELIDNNGNSLKIRRDSSGMPRHLLMPDNQIITLTVGTNGGLKVVSTQNLELGLMTYDGNTGLLATKSDETGWTTFYDYDHEGRLTNVTRPTGVVTSLHREMEKSITIDIENSNRDDDVTVITNLSSVEASYTVVQDQVRNSYQLCNNGTLRVMYANGMGISFHSEPHVLAGTITPTIGRCNISLPMENGLNSIEWRLRKEQIKGKVTIFGRKLRVHGRNLLSIDYDRSIRTEKIYDDHRKFTLRIIYDQVGRPFLWLPSSGLAAVNVSYFFNGRLAGLQRGAMSERTDIDKQGRIVSRMFADGKVWSYSYLDKSMVLLLQSQRQYIFEYDSSDRLLAVTMPSVARHSMSTHTSIGYIRNIYNPPESNASVIFDYSDDGRILKTSFLGTGRQVFYKYGKLSKLSEIVYDSTAVTFGYDETTGVLKMVNLQSGGFSCTIRYRKIGPLVDKQIYRFSEEGMVNARFDYTYHDNSFRIASIKPVISETPLPVDLYRYDEISGKVEHFGKFGVIYYDINQIITTAVMTLSKHFDTHGRIKEVQYEMFRSLMYWMTVQYDSMGRVIKRELKLGPYANTTKYTYDYDGDGQLQSVAVNDRPTWRYSYDLNGNLHLLNPGNSVRLMPLRYDLRDRITRLGDVQYKIDDDGYLCQRGSDIFEYNSKGLLTRAYNKASGWSVQYRYDGVGRRASYKTNLGHHLQYFYSDLHNPTRITHVYNHSNSEITSLYYDLQGHLFAMESSSGEEYYVASDNTGTPLAVFSINGLMIKQLQYTAYGEIYYDSNPDFQMVIGFHGGLYDPLTKLVHFTQRDYDVLAGRWTSPDYTMWKNVGKEPAPFNLYMFKSNNPLSSELGLKNYVTDVKSWLVMFGFQLSNIIPGFPRAKMYFVPPPYELSESQASENGQLITGVQQKTERHNQAFMALEGQVITKKLHASIREKAGHWFATTTPIIGKGIMFAIKEGRVTTGVSSIASEDSRKVASVLNNAYYLDKMHYSIEGKDTHYFVKIGSADGDLVTLGTTIGRKVLESGVNVTVSQPTLLVNGRTRRFTNIEFQYSTLLLSIRYGLTPDTLDEEKARVLDQARQRALGTAWAKEQQKARDGREGSRLWTEGEKQQLLSTGRVQGYEGYYVLPVEQYPELADSSSNIQFLRQNEMGKR

SEQ ID NO: 7 - protein, 97 aa, Homo sapiens
DAGYKSLLKITMTQSTVPLNLIRVHLMVAVEGHLFQKSFQASPNLAYTFIWDKTDAYGQRVYGLSDAVVSVGFEYETCPSLILWEKRTALLQGFELD

SEQ ID NO: 8 - protein, 120 aa, Homo sapiens
MDLSGFVRPDPVIISSPLSTFFSDAGYKSLLKITMTQSTVPLNLIRVHLMVAVEGHLFQKSFQASPNLAYTFIWDKTDAYGQRVYGLSDAVVSVGFEYETCPSLILWEKRTALLQGFELD

SEQ ID NO: 9 - protein, 120 aa, Homo sapiens
TQVLHEEIELPGSNVKLRYLSSRTAGYKSLLKITMTQSTVPLNLIRVHLMVAVEGHLFQKSFQASPNLAYTFIWDKTDAYGQRVYGLSDAVVSVGFEYETCPSLILWEKRTALLQGFELD

SEQ ID NO: 10 - protein, 120 aa, Homo sapiens
TQVLHEEIEVPGSSIKLIYLSSRTAGYKSLLKIIMTQSLVPLNLIKVHLMVAVEGHLFQKSFLASPNLAYTFIWDKTDAYGQKVYGLSDAVVSVGFEYETCPSLILWEKRTALLQGFELD

SEQ ID NO: 11 - protein, 96 aa, Homo sapiens
TQIVQESIQIPGSDLHLTYQSSQASGYLSIVRMRLTAETIPPTLTHVHVGVEIEGALHVKTYEADPSLVHTFAWNKRNVYRQKVYGVTVARISVGY

SEQ ID NO: 12 - protein, 98 aa, Homo sapiens
SQVIQESLQIPGTGLNLVYHSSRAAGYLSTIKLQLTPDVIPTSLHLIHLRITIEGILFERIFEADPGIKFTYAWNRLNIYRQRVYGVTTAVVKVGYQY

SEQ ID NO: 13 - protein, 7 aa, Homo sapiens
IMTQSLV

SEQ ID NO: 14 - protein, 7 aa, Homo sapiens
RLTAETI

SEQ ID NO: 15 - protein, 7 aa, Homo sapiens
QLTPDVI

SEQ ID NO: 16 - protein, 96 aa, Homo sapiens
TQVLHEEIELPGSNVKLRYLSSRTAGYKSLLKITMTQSTVPLNLIRVHLMVAVEGHLFQKSFQASPNLAYTFIWDKTDAYGQRVYGLSDAVVSVGF

SEQ ID NO: 17 - protein, 8 aa, Homo sapiens
TMTQSTVP